(19) United States
(12) Patent Application Publication
(10) Pub. No.: US 2026/0308194 A1
(43) Pub. Date: Oct. 8, 2026

(54) HUMAN MILK OLIGOSACCHARIDE ANALOGS CONTAINING N-ACETYLMANNOSAMINE

(71) Applicant: The Government of the United States of America, as represented by the Secretary of the Navy, Arlington, VA (US)

(72) Inventors: Ryan W. Penhallurick, Washington, DC (US); Adam A. Meares, Burke, VA (US); Gregory A. Ellis, Rockville, MD (US); Igor L. Medintz, Springfield, VA (US)

(21) Appl. No.: 19/342,735

(22) Filed: Sep. 29, 2025

Related U.S. Application Data
(60) Provisional application No. 63/783,394, filed on Apr. 4, 2025.

(51) Int. Cl. C07H 5/06

(52) U.S. Cl. CPC C07H 5/06

(57) ABSTRACT

Disclosed is a synthetic human milk oligosaccharide (HMO) analog containing a core composed of N-acetylmannosamine (ManNAc) and lactose (Lac).

### CROSS-REFERENCE TO RELATED APPLICATIONS

This patent application claims priority to U.S. Provisional Patent Application Ser. No. 63/783,394, filed Apr. 4, 2025; which is incorporated by reference herein in its entirety into this disclosure.

### FEDERALLY-SPONSORED RESEARCH AND DEVELOPMENT

The United States Government has ownership rights in this subject disclosure. Licensing inquiries may be directed to Office of Technology Transfer, US Naval Research Laboratory, Code 1004, 4555 Overlook Avenue SW, Washington, DC 20375, USA; +1.202.767.7230; techtran@nrl.navy.mil, referencing NC 212005.

### BACKGROUND OF THE SUBJECT DISCLOSURE

### Field of the Subject Disclosure

The present subject disclosure relates generally to synthesis of analogs of human milk oligosaccharides (HMOs). More particularly, the present subject disclosure relates to methods for forming synthetic human milk oligosaccharides (HMOs) with a N-acetylmannosamine containing core.

### Background of the Subject Disclosure

Human milk oligosaccharides (HMOs) constitute a variety of short-chain carbohydrates contained in human breast milk. Natural HMOs are comprised of glucose (Glc), N-acetylglucosamine (GlcNAc), galactose (Gal), N-acetylneuraminic acid (sialic acid, Neu5Ac) and fucose (Fuc) constituent monomers.¹ The HMO core starts with lactose (Gal-β1,4-Glc) at the reducing end, which is elongated by β1,3-GlcNAc (producing core LNTII, GlcNAc-β1,3-Lac). Lacto-N-tetraose (type I, LNT) or lacto-N-neotetraose (type II, LNnT) are subsequently formed via 1,3- or β1,4-galactosylation, respectively, of the GlcNAc constituent to complete the core. These backbones are then further functionalized to compose the wide array of HMOs present in breast milk.

HMOs have been shown to play important roles in infant gut health and immune system development and may reduce infectious disease risk.¹ Furthermore, some HMOs have been identified for commercial use as possible therapeutic and prebiotic agents.² For example, the trisaccharide lacto-N-triose II (LNTII; GlcNAc-β1,3-Lac), which comprises the backbone of both type I and type II HMOs, may assist in crosstalk between commensal bacteria and human gut epithelial cells,³ while the tetrasaccharide lacto-N-tetraose (LNT; Gal-31,3-GlcNAc-β1,3-Lac) may have potential as a rotavirus inhibitor.⁴ Fucosylated HMOs have shown promise as norovirus and rotavirus spike inhibitors,⁵ while certain sialylated HMOs have been shown to be important in supporting immune system and neurologic health⁶ and are hypothesized to have potential as influenza⁷ and SARS-COV-2 antivirals.⁸

A need exists for a synthetic HMO which emulates the properties of natural HMOs without suffering from the limitations of natural HMOs.

### SUMMARY OF THE SUBJECT DISCLOSURE

The present subject disclosure relates generally to synthetic core HMOs. The wide range of antiviral and therapeutic efficacy of HMOs has potential to enhance warfighter performance, and protect and treat warfighter dependents.

While naturally occurring HMOs contain N-acetylglucosamine along the backbone, substitution with other N-acetylhexosamines may provide enhanced therapeutic and pre-/probiotic potential compared with that of their natural counterparts. For example, mannose and N-acetylmannosamine containing oligosaccharides are important in gut microbial functioning, with mannan-rich mixtures currently being used as prebiotic supplements for livestock and show potential for equivalent use in humans.⁹ GlcNAc-Lac (LNTII) is a known naturally occurring core trisaccharide in many human breast milk HMOs¹⁰ and GalNAc-Lac has been synthesized previously;¹¹ however, synthesis of ManNAc-Lac has yet to be described. The synthesis, purification and characterization of a novel HMO analog is described herein, characterized by substitution of N-acetylglucosamine in the backbone of lacto-N-triose II with N-acetylmannosamine (ManNAc) to form ManNAc-Lac (ManNAc-α- or β1,3-Gal-β1,4-Glc). Functionalization of this ManNAc-Lac core to form ManNAc-containing HMO analogs consisting of R¹, R² and R³ of the ManNAc constituent and R⁴ of the lactose (Lac) constituent (FIG. 1) is also described, and wherein R¹ is H or α2,6-Neu5Ac, R² is H, α1,4-Fuc, β1,4-Gal or β1,4-Gal-α2,3-Neu5Ac, R³ is H, α1,3-Fuc, β1,3-Gal or β1,3-Gal-α2,3-Neu5Ac and R⁴ is H or α1,3-Fuc; and wherein the ManNAc constituent cannot be directly (attached to) and simultaneously fucosylated (Fuc) and sialylated (Neu5Ac) or multifucosylated.

ManNAc-Lac (R¹, R², R³, R⁴=H) was chemoenzymatically synthesized using modifications to methods of synthesis of the natural HMO core LNTII (GlcNAc-Lac). Enzyme-catalyzed biosynthesis of LNTII (GlcNAc-Lac) has been accomplished via transglycosylation with β-N-acetylhexosaminidases using an activated GlcNAc and lactose as substrates.¹¹ A screen of enzymes showed promise for efficient formation of LNTII via Bifidobacterium bifidum β-N-acetylhexosaminidase I (BbhI), which was able to produce both GlcNAc-Lac and GalNAc-Lac using 4-nitrophenyl N-acetylglucosamine (pNP) as an activated precursor.¹¹ Undesirably, BbhI was also shown to catalyze hydrolysis of LNTII back to GlcNAc and lactose. To minimize this hydrolysis, a mutant D746E BbhI was identified, which catalyzed efficient production of LNTII using an oxazoline-activated GlcNAc substrate (GlcNAc-oxa).¹²

For this subject disclosure, ManNAc was first activated to N-acetylmannosamine 1,2-oxazoline (ManNAc-oxa).¹³ D746E BbhI was then added to a buffered solution containing ManNAc-oxa and lactose to form ManNAc-Lac. It should be noted that while a β-N-acetylhexosaminidase was utilized here, it is proposed that higher yield may be achieved using α-N-acetylhexosaminidase or other engineered N-acetylhexosaminidases. Unreacted lactose was digested with lactase (Kluyveromyces lactis β-galactosidase) and unreacted activated precursors were quenched and removed using flash activated charcoal (Flash-AC) chromatography. ManNAc-Lac was isolated using flash hydrophilic interaction (Flash-HILIC) chromatography and lyophilized.

Functionalization of the natural GlcNAc-Lac core occurs in a variety of other HMOs.^{14, 15} However, functionalized HMOs with a non-GlcNAc containing core have yet to be synthesized or discovered. Functionalization of the ManNAc-Lac core is hypothesized to be achieved via analogous enzyme-catalyzed reactions. Galactosylation at position R³ to form Gal-ManNAc-Lac may be accomplished using ManNAc-Lac and uridine diphosphate galactose (UDP-Gal) as substrates in the presence of β1,3-galactosyltransferase (β3GalT), analogous to a previously-published synthetic strategy to LNT (Gal-GlcNAc-Lac).¹⁴ In the presence of guanosine diphosphate fucose (GDP-Fuc), ManNAc-Lac core oligosaccharides can be analogously fucosylated at R², R³ and/or R⁴ with α1,3/4-fucosyltransferases.¹⁵ In the presence of cytidine monophosphate N-acetylneuraminic acid (CMP-Neu5Ac), ManNAc-Lac core oligosaccharides can be sialylated at R² or R³ with α2,3-sialyltransferase and α2,6-sialyltransferase, respectively, analogous to sialylation of natural GlcNAc containing core HMOs. All functionalized products are hypothesized here to be isolated using flash size-exclusion chromatography (Flash-SEC) or flash ion-exchange chromatography (Flash-IEC) and lyophilized for purification.

In one exemplary embodiment, the present subject disclosure is a human milk oligosaccharide (HMO) analog. The analog includes N-acetylmannosamine (ManNAc) and lactose (Lac).

In another exemplary embodiment, the present subject disclosure is a method comprising formulating a human milk oligosaccharide (HMO) analog using the scheme depicted in FIG. 2.

### BRIEF DESCRIPTION OF THE DRAWINGS

The patent or application file may contain at least one drawing executed in color. Copies of this patent or patent application publication with color drawing(s) will be provided by the Office upon request and payment of the necessary fee.

FIG. 1 shows a Markush structure of the proposed chemical subject disclosure, according to an exemplary embodiment of the present subject disclosure.

FIG. 2 depicts a chemoenzymatic synthetic scheme for the novel oligosaccharide core ManNAc-Lac (R¹, R², R³, R⁴=H), according to an exemplary embodiment of the present subject disclosure.

FIG. 3A shows a representative time series quantitative NMR ¹H spectra for the β-N-acetylhexosaminidase I (BbhI) assay with N-acetylmannosamine 1,2-oxazoline (ManNAc-oxa) with full spectra shown for reactions using ManNAc-oxa as substrate, according to an exemplary embodiment of the present subject disclosure.

FIG. 3B shows a representative time series quantitative NMR ¹H spectra for the β-N-acetylhexosaminidase I (BbhI) assay with N-acetylmannosamine 1,2-oxazoline (ManNAc-oxa) with enlarged anomeric proton peak shown for reactions using ManNAc-oxa as substrate, according to an exemplary embodiment of the present subject disclosure.

FIG. 3C shows a representative time series quantitative NMR ¹H spectra for the β-N-acetylhexosaminidase I (BbhI) assay with N-acetylmannosamine 1,2-oxazoline (ManNAc-oxa), shown with integration of the anomeric hydrogen peak relative to an internal standard used to quantify the time-dependent change in oxazoline concentration, according to an exemplary embodiment of the present subject disclosure.

FIG. 4A depicts kinetics of D746E BbhI catalyzed N-acetylmannosamine 1,2-oxazoline activated transglycosylation using Michaelis-Menten plot, according to an exemplary embodiment of the present subject disclosure.

FIG. 4B depicts kinetics of D746E BbhI catalyzed N-acetylmannosamine 1,2-oxazoline activated transglycosylation using Lineweaver-Burk plots, according to an exemplary embodiment of the present subject disclosure.

FIG. 5 depicts chemoenzymatic synthesis of ManNAc-Lac via N-acetylated sugar oxazoline activation with D746E BbhI, according to an exemplary embodiment of the present subject disclosure.

FIG. 6A depicts mass spectrum of ManNAc-Lac, according to an exemplary embodiment of the present subject disclosure.

FIG. 6B depicts silica-gel thin-layer chromatograph confirming production of ManNAc-Lac, according to an exemplary embodiment of the present subject disclosure.

FIG. 7A depicts (in color) exemplary structural representations of ManNAc-Lac core Type I HMO analogs, according to an exemplary embodiment of the present subject disclosure.

FIG. 7B depicts (in black/white) exemplary structural representations of ManNAc-Lac core Type I HMO analogs, according to an exemplary embodiment of the present subject disclosure.

FIG. 8A depicts (in color) exemplary structural representations of ManNAc-Lac core Type II HMO analogs, according to an exemplary embodiment of the present subject disclosure.

FIG. 8B depicts (in black/white) exemplary structural representations of ManNAc-Lac core Type II HMO analogs, according to an exemplary embodiment of the present subject disclosure.

### DETAILED DESCRIPTION OF THE SUBJECT DISCLOSURE

The present subject disclosure addresses the shortcomings of natural HMOs, as discussed above.

### Definitions

Before describing the present subject disclosure in detail, it is to be understood that the terminology used in the specification is for the purpose of describing particular embodiments, and is not necessarily intended to be limiting. Although many methods, structures and materials similar, modified, or equivalent to those described herein can be used in the practice of the present subject disclosure without undue experimentation, the preferred methods, structures and materials are described herein. In describing and claiming the present subject disclosure, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Purposes and Description of Function

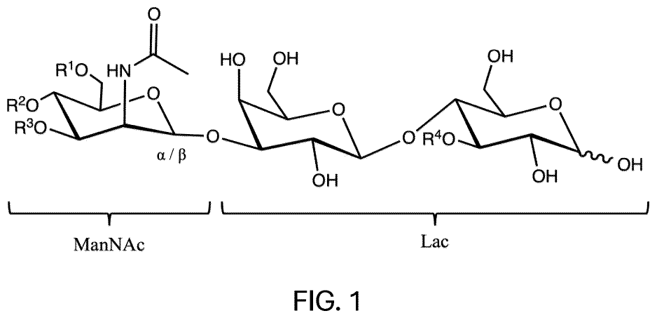

Human milk oligosaccharides (HMOs) play important roles in infant gut health and immune system development and may reduce infectious disease risk. Various HMOs, especially those containing fucosyls and sialyls, have commercial potential as livestock and human pre/probiotics, antivirals and as pharmaceuticals in treating immunological or neurological disorders. Furthermore, mannose and N-acetylmannosamine (ManNAc) containing oligosaccharides have shown potential as pre/probiotics and therapeutics. 16 Here, we have designed, synthesized and isolated a novel HMO analog core characterized by substitution of N-acetylglucosamine in the backbone of LNTII with N-acetylmannosamine (ManNAc) to form ManNAc-Lac (ManNAc-α1,3 or β1,3-Gal-β1,4-Glc). Functionalization of this ManNAc-Lac core to form ManNAc-containing HMO analogs consisting of R¹, R² and R³ of the ManNAc constituent and R⁴ of the lactose (Lac) constituent (FIG. 1) is also described, and wherein R¹ is H or α2,6-Neu5Ac, R² is H, α1,4-Fuc, β1,4-Gal or β1,4-Gal-α2,3-Neu5Ac, R³ is H, α1,3-Fuc, β1,3-Gal or β1,3-Gal-α2,3-Neu5Ac and R⁴ is H or α1,3-Fuc; and wherein the ManNAc constituent cannot be directly (attached to) and simultaneously fucosylated and sialylated or multifucosylated.

FIG. 1 depicts the Markush structure of the proposed subject disclosure described herein. The N-acetylmannosamine (ManNAc) and lactose (Lac) constituents of the ManNAc-Lac core are identified. As shown in FIG. 1, a novel Markush structure of the proposed chemical subject disclosure described herein is presented, with N-acetylmannosyl (ManNAc) and lactosyl (Lac) constituents of the core HMO analog structure being identified. R¹ is H or α2,6-Neu5Ac, R² is H, α1,4-Fuc, β1,4-Gal or β1,4-Gal-α2,3-Neu5Ac, R³ is H, α1,3-Fuc, β1,3-Gal or β1,3-Gal-α2,3-Neu5Ac and R⁴ is H or α1,3-Fuc; wherein the ManNAc constituent cannot be directly (attached to) and simultaneously fucosylated and sialylated or multifucosylated.

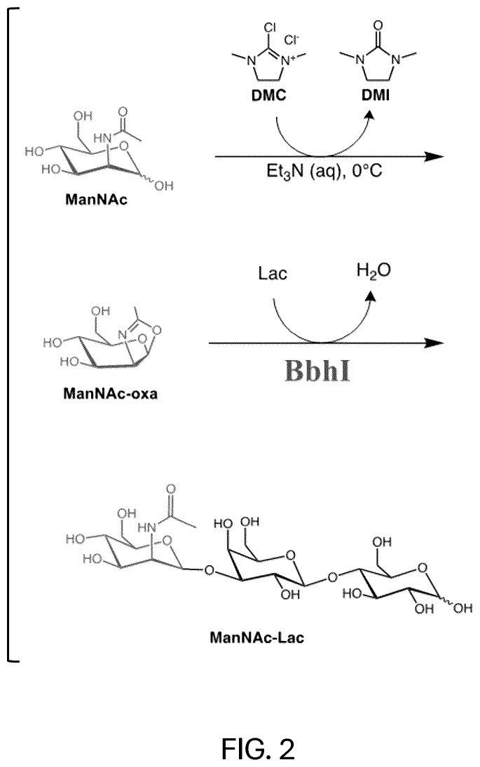

FIG. 2 depicts the chemoenzymatic reaction scheme of ManNAc-Lac (R¹, R², R³, R⁴=H) formation. ManNAc was first activated to N-acetylmannosamine 1,2-oxazoline (ManNAc-oxa).¹³ Mutant D746E Bifidobacterium bifidum β-N-acetylhexosaminidase I (BbhI) was then added to a buffered solution containing ManNAc-oxa and lactose to form ManNAc-Lac. As shown in FIG. 2, a novel chemoenzymatic synthetic scheme for the novel oligosaccharide core ManNAc-Lac (R¹, R², R³, R⁴=H) is depicted. ManNAc constituent highlighted are shown in green. Abbreviations: N-acetylmannosamine, ManNAc; 2-chloro-1,3-dimethylimidazolinium chloride, DMC; 1,3-dimethyl-2-imidazolidinone, DMI; triethylamine, Et₃N; N-acetylmannosamine 1,2-oxazoline, ManNAc-oxa; Bifidobacterium bifidum β-N-acetylhexosaminidase I, BbhI; lactose, β-D-galactosyl-(1→4)-D-glucose, Lac. O-2-(acetylamino)-2-deoxy-D-mannopyranosyl-(1→3)-O-β-D-galactopyranosyl-(1→4)-D-glucose, ManNAc-Lac.

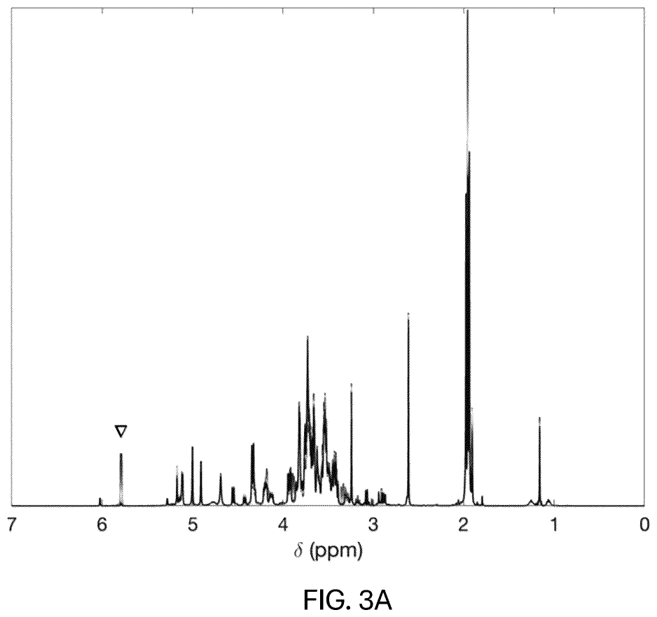

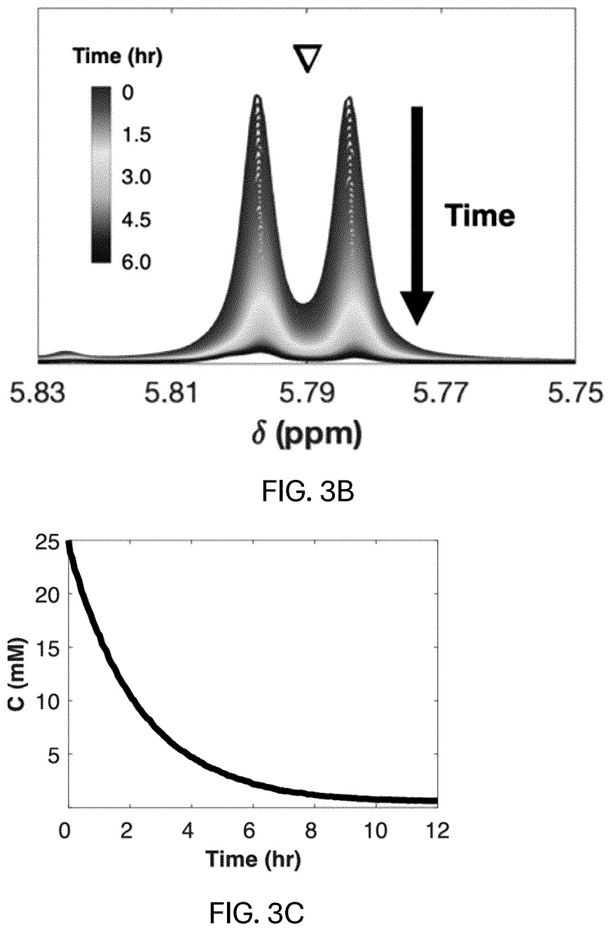

FIG. 3 shows enzyme-catalyzed consumption of ManNAc-oxa monitored using quantitative NMR (qNMR). More specifically, FIG. 3 shows a representative time series quantitative NMR ¹H spectra for the β-N-acetylhexosaminidase I (BbhI) assay with N-acetylmannosamine 1,2-oxazoline (ManNAc-oxa). FIG. 3A shows a full spectra and FIG. 3B shows enlarged anomeric proton peak for reactions using ManNAc-oxa as substrate. The decrease in peak intensity over time (red to blue) corresponds to oxazoline consumption. Anomeric hydrogen peak denoted V. Integration of the anomeric hydrogen peak relative to an internal standard is used to quantify the time-dependent change in oxazoline concentration, shown in FIG. 3C. Reaction conditions: 50 nM D746E BbhI, 25 mM HexNAc-oxa, 30 mM lactose, 20 mM potassium hydrogen phthalate (KHP) in 100 mM sodium phosphate buffer (pH=7.5, 10% D₂O) at 25° C. A multiple peak NOESY suppression pulse program was used to suppress the water and a/β aliphatic triethylamine peaks.

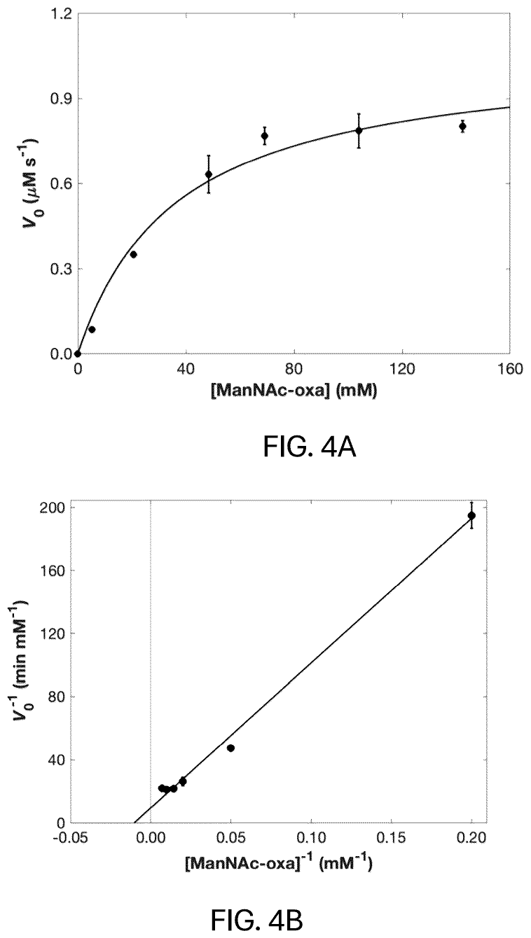

FIG. 4 shows initial rates of ManNAc-oxa consumption were used to determine apparent kinetic parameters for enzyme catalyzed production of ManNAc-Lac. The Michaelis constant K_{M} and turnover K_{cat} were determined to be 36.6±9.0 mM and 10.7±0.9 s⁻¹, respectively. Upon reaction completion, unreacted lactose was digested with lactase (Kluyveromyces lactis β-galactosidase) and unreacted activated precursors were quenched and removed using flash activated charcoal (Flash-AC) chromatography. More specifically, FIG. 4 depicts kinetics of D746E BbhI catalyzed N-acetylmannosamine 1,2-oxazoline activated transglycosylation. FIG. 4A depicts Michaelis-Menten and FIG. 4B depicts Lineweaver-Burk plots. Reaction conditions: 100 nM D746E BbhI, 0-140 mM ManNAc-oxa, 250 mM lactose, 20 mM potassium hydrogen phthalate (KHP) in 100 mM sodium phosphate buffer (pH=7.5, 10% D₂O) at 37° C.

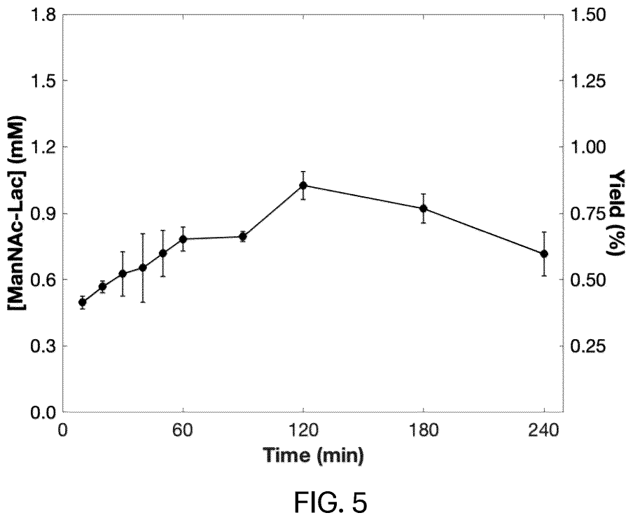

FIG. 5 shows progress of ManNAc-Lac synthesis monitored using HILIC-LC/MS. More specifically, FIG. 5 depicts chemoenzymatic synthesis of ManNAc-Lac via N-acetylated sugar oxazoline activation with D746E BbhI. Progress curve quantified from HILIC-LC/MS analysis. Reactions contained 120 mM HexNAc-oxa, 150 mM lactose, 100 nM D746E BbhI in Tris-HCl (pH=7.5) at 37° C.

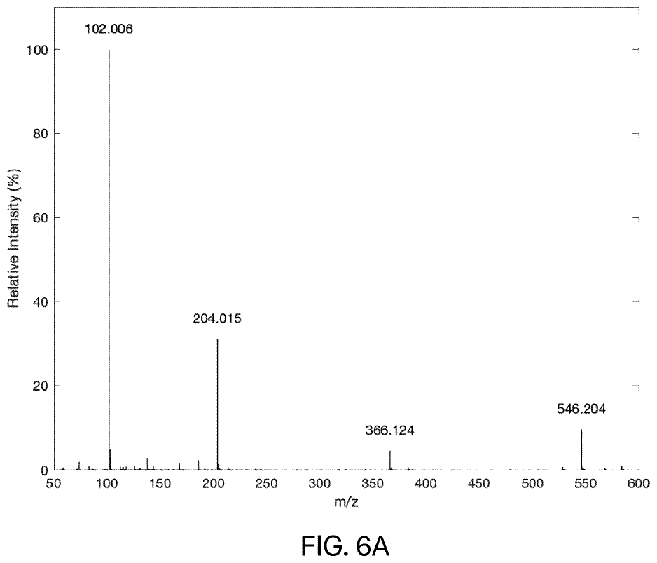

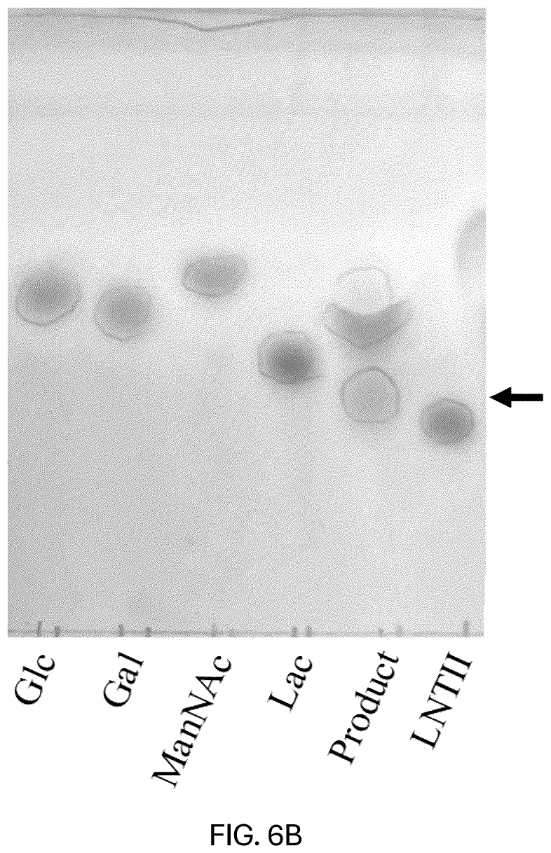

FIG. 6 shows validation of synthesis of ManNAc-Lac confirmed via HILIC-LC/MS ([M+H]⁺=546.20) and thin-layer chromatography. More specifically, FIG. 6A depicts mass spectrum of ManNAc-Lac. Product peak: [M+H]⁺=546.204. Fragmentation peaks: lactose, [M+Na]⁺=366.124; mannose, [M+Na]⁺=204.015; 2-iminobutyric acid, [M+H]⁺=102.006. FIG. 6B depicts silica-gel thin-layer chromatograph confirming production of ManNAc-Lac. Spots (left to right): lactose, reaction, LNTII (GlcNAc-Lac). Mobile phase was 2:1:1 1-butanol:acetic acid:water. TLC stained with thymol-sulfuric acid stain and heated at 150° C. for approx. 45 min.

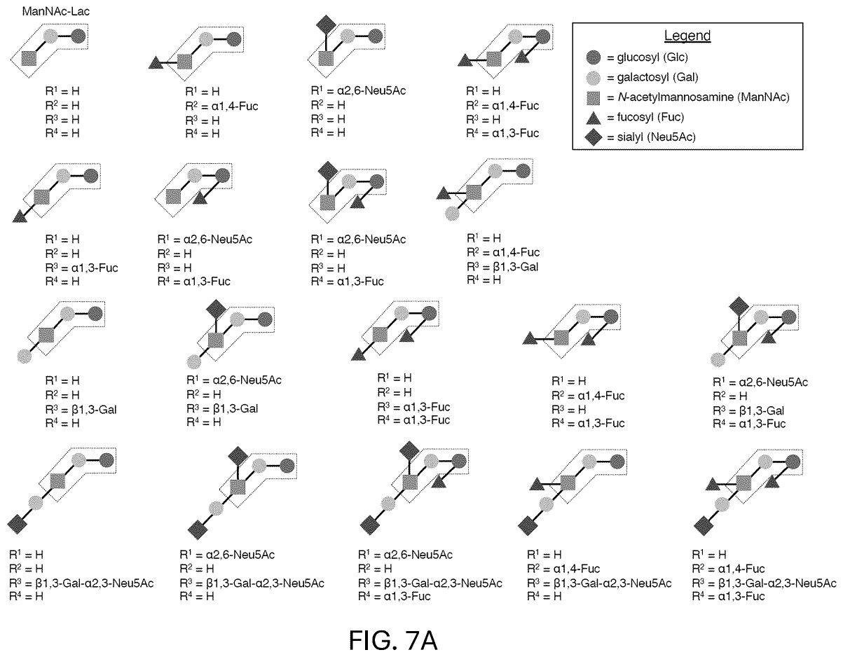

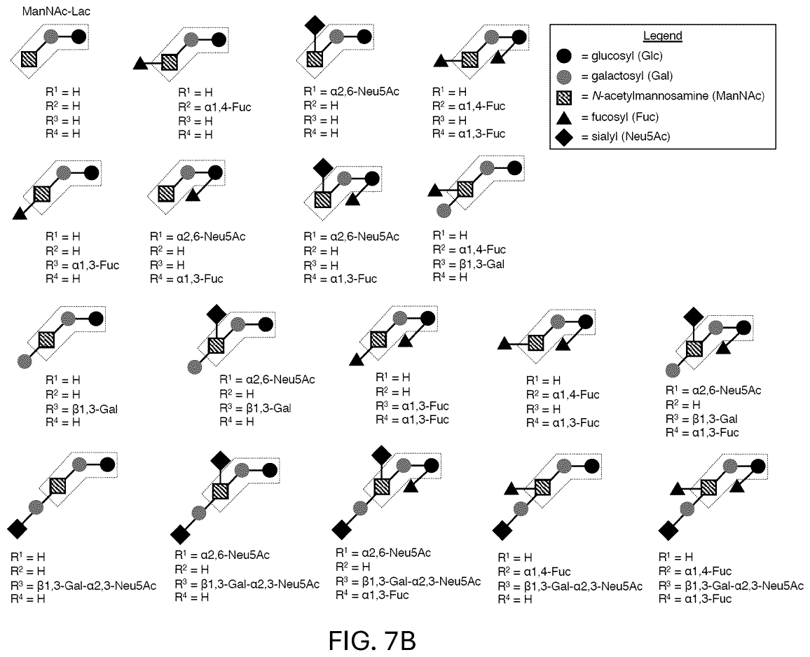

FIG. 7 depicts enzyme catalyzed galactosylation at position R³, fucosylation at R², R³ or R⁴ and sialylation at R² or R³ will result in a variety of novel HMO analogs containing the ManNAc-Lac core described herein. FIGS. 7A (color) and 7B (black and white) depict exemplary Type I HMO analogs, with the ManNAc-Lac core highlighted in each structure.

More specifically, FIG. 7A depicts (in color) exemplary structural representations of ManNAc-Lac core Type I HMO analogs. ManNAc-Lac core of each analog is encircled by dashed lines. Hexoses and N-acetylhexosamines represented by circles and squares, respectively. Glucosyl, galactosyl and mannosyl monomers represented by blue, yellow and green, respectively. Fucosyl (Fuc) and sialyl (Neu5Ac) monomers represented by red triangles and purple diamonds, respectively. Identities of R¹⁻⁴ provided under each depiction.

FIG. 7B depicts (in black/white) exemplary structural representations of ManNAc-Lac core Type I HMO analogs. ManNAc-Lac core of each analog is encircled by dashed lines. Glucosyl (Glc) and galactosyl (Gal) monomers represented by dark and light circles, respectively. N-acetylmannosyl (ManNAc) represented by striped square. Fucosyl (Fuc) and sialyl (Neu5Ac) monomers represented by triangles and diamonds, respectively. Identities of R¹⁻⁴ provided under each depiction.

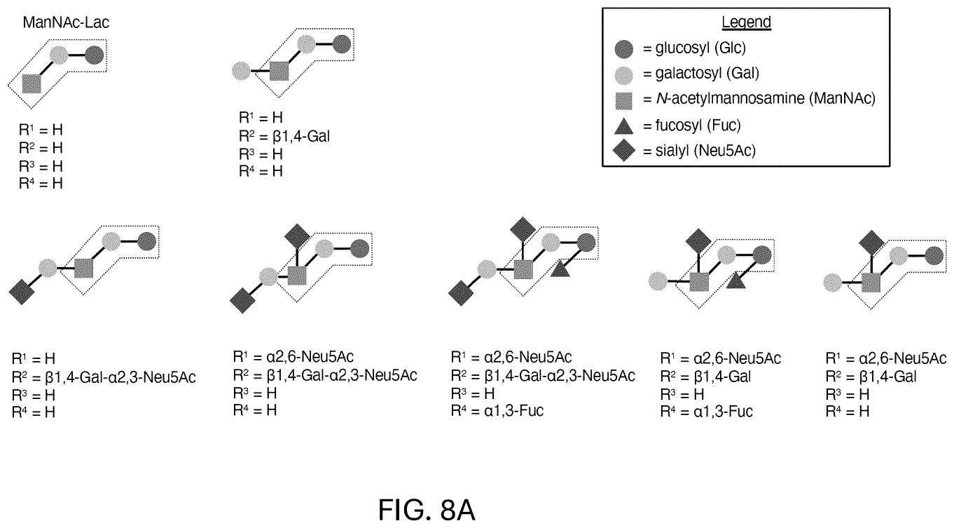

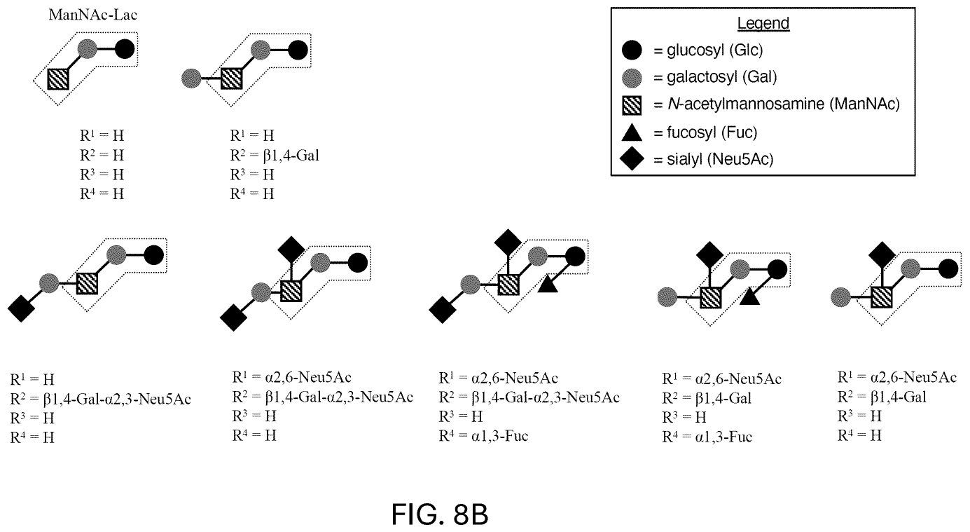

FIGS. 8A (color) and 8B (black and white) depict exemplary Type II HMO analogs, with the ManNAc-Lac core highlighted in each structure. More specifically, FIG. 8A depicts (in color) exemplary structural representations of ManNAc-Lac core Type II HMO analogs. ManNAc-Lac core of each analog is encircled by dashed lines. Hexoses and N-acetylhexosamines represented by circles and squares, respectively. Glucosyl, galactosyl and mannosyl monomers represented by blue, yellow and green, respectively. Fucosyl (Fuc) and sialyl (Neu5Ac) monomers represented by red triangles and purple diamonds, respectively. Identities of R¹⁻⁴ provided under each depiction.

FIG. 8B depicts (in black/white) exemplary structural representations of ManNAc-Lac core Type II HMO analogs. ManNAc-Lac core of each analog is encircled by dashed lines. Glucosyl (Glc) and galactosyl (Gal) monomers represented by dark and light circles, respectively. N-acetylmannosyl (ManNAc) represented by striped square. Fucosyl (Fuc) and sialyl (Neu5Ac) monomers represented by triangles and diamonds, respectively. Identities of R¹⁻⁴ provided under each depiction.

The exemplary Type I and Type II HMO analogs shown in FIGS. 7A-7B and 8A-8B, respectively, are not limiting of the present subject disclosure. Other Type I (not shown) or Type II (not shown) HMO analogs, as recognized and appreciated by one having ordinary skill in the art after consideration of the present subject disclosure, are also within the scope of the present disclosure. Galactosylation to form Gal-ManNAc-Lac can be accomplished using ManNAc-Lac and uridine diphosphate galactose (UDP-Gal) as substrates in the presence of β-1,3-galactosyltransferase (β3GalT), analogous to a previously-published synthetic strategy to LNT (Gal-GlcNAc-Lac).¹⁴ In the presence of guanosine diphosphate fucose (GDP-Fuc), ManNAc-Lac core oligosaccharides can be fucosylated at R², R³ and/or R⁴ with α1,3/4-fucosyltransferases.¹⁵ In the presence of cytidine monophosphate N-acetylneuraminic acid (CMP-Neu5Ac), ManNAc-Lac core oligosaccharides can be sialylated at R² or R³ with α2,3-sialyltransferase and α2,6-sialyltransferase, respectively. All functionalized products can be isolated using flash size-exclusion chromatography (Flash-SEC) or flash ion-exchange chromatography (Flash-IEC) and lyophilized for purification.

### Detailed Description of Chemoenzymatic Synthesis of ManNAc-Lac

N-acetylmannosamine 1,2-oxazoline (ManNAc-oxa) was synthesized using a modified procedure.¹³ Briefly, 250 mM ManNAc, 750 mM 2-chloro-1,3-dimethylimidazolinium chloride (DMC) and 2.25 M triethylamine reacted at 0° C. in aqueous solvent for 30 minutes with stirring. The reaction mixture was washed with 1:1.5 (v/v) cold dichloromethane to remove 1,3-dimethyl-2-imidazolidinone (DMI) and unreacted DMC. Flash activated charcoal chromatography (Flash-AC) was used for an initial removal of impurities. The 220 nm absorbing fractions were pooled and lyophilized for ~36 hours. Solid reaction mixture was desalted using cold 9.5:1 neat acetonitrile to crude (w/w) with stirring at 4° C. for 15 minutes. The mixture was then vacuum filtered with filtrate initially dried via short-path distillation followed by lyophilization overnight. Oxazoline conversion was confirmed with silica gel thin-layer chromatography (TLC) stained with potassium permanganate and ¹H NMR (D20, 400 MHz). The weight percent of each sugar oxazoline was determined by quantitative NMR (qNMR) with potassium hydrogen phthalate (KHP) as an internal standard.¹⁷

Enzyme-catalyzed synthesis of ManNAc-Lac was performed using a modified procedure.¹¹ 150 mM lactose monohydrate and 120 mM ManNAc-oxa were dissolved in 100 mM sodium phosphate buffer (pH=7.5). Formation of ManNAc-Lac began upon addition of 100 nM D746E BbhI at 37° C. with 120 rpm shaking. The reaction was stopped after 120 min with addition of 1.5×methanol (v/v), incubation at 4° C., and centrifugation. MeOH was removed via short-path distillation. 500 U mL-1 lactase (Kluyveromyces lactis β-galactosidase) was added to supernatant at 37.1° C. with 120 rpm shaking to break down unreacted lactose. The reaction was once again quenched with 1.5× methanol (v/v), incubated, centrifuged, and supernatant was collected. Product formation was confirmed with silica gel TLC (2:1:1 1-butanol:acetic acid:water (v/v/v) mobile phase; stained with thymol-sulfuric acid; heated at 150° C. for 45 minutes) and mass spectrometry. A maximum yield of ~1% was observed after 120 min, as determined by LC/MS.

### Advantages and New Features

Herein, this chemical subject disclosure, ManNAc-Lac, and the subsequent ManNAc-Lac core containing HMO analogs have many possible applications and advantages over other pre-probiotics and natural HMO derived therapeutics. Such features include, but are not limited to:
(1) Substitution of N-acetylglucosamine for N-acetylmannosamine may present stronger binding with commensal gut microbes which naturally use mannosyl containing oligosaccharides.
(2) HMO analogs that act as antibiotics may exhibit enhanced binding to target bacterial enzymes due to the presence of the N-acetylmannosyl in the HMO analog core.
(3) The mechanisms of action and binding of these HMO analogs will likely be similar to that of natural HMO counterparts; however, the presence of N-acetylmannosyl within the core structure may slow degradation by human carbohydrases, thereby extending the therapeutic half-life of these sugars.
(4) Presence of an N-acetylmannosyl may lead to enhanced binding to target enzymes when used as an antiviral or therapeutic.
(5) Galactosylation, fucosylation and/or sialylation may lead to similar properties as the natural HMO counterparts, while retaining the unique properties contained in the N-acetylmannosyl core.
(6) Antibody or protein transglycosylation of these HMO analogs may desirably alter the functionality of the target.

### Alternatives

Natural human milk oligosaccharides have a variety of beneficial properties. Other mannan-rich, naturally derived prebiotics are available commercially; however, these do not exhibit the unique properties of HMOs. Targeted therapeutics as antibiotics or antivirals have been developed.

### REFERENCES

The following references, cited within this disclosure, are incorporated by reference herein in their entirety into this disclosure:
(1) Wiciński, M.; Sawicka, E.; Gębalski, J.; Kubiak, K.; Malinowski, B. Human milk oligosaccharides: Health benefits, potential applications in infant formulas, and pharmacology. Nutrients 2020, 12 (1).
(2) Bode, L. Human milk oligosaccharides: Every baby needs a sugar mama. Glycobiology 2012, 22 (9), 1147.
(3) Kong, C. L.; Cheng, L. H.; Krenning, G.; Fledderus, J.; de Haan, B. J.; Walvoort, M. T. C.; de Vos, P. Human milk oligosaccharides mediate the crosstalk between intestinal epithelial caco-2 cells and Lactobacillus plantarum WCFS1 in an in vitro model with intestinal peristaltic shear force. J. Nutr. 2020, 150 (8), 2077.
(4) Morozov, V.; Hansman, G.; Hanisch, F. G.; Schroten, H.; Kunz, C. Human milk oligosaccharides as promising antivirals. Mol. Nutr. Food Res. 2018, 62 (6), 1700679.
(5) Weichert, S.; Koromyslova, A.; Singh, B. K.; Hansman, S.; Jennewein, S.; Schroten, H.; Hansman, G. S. Structural basis for norovirus inhibition by human milk oligosaccharides. J. Virol. 2016, 90 (9), 4843.
(6) Hobbs, M.; Jahan, M.; Ghorashi, S. A.; Wang, B. Current perspective of sialylated milk oligosaccharides in mammalian milk: Implications for brain and gut health of newborns. Foods 2021, 10 (2). Kulinich, A.; Liu, L. Human milk oligosaccharides: The role in the fine-tuning of innate immune responses. Carb. Res. 2016, 432, 62. Masi, A. C.; Embleton, N. D.; Lamb, C. A.; Young, G.; Granger, C. L.; Najera, J.; Smith, D. P.; Hoffman, K. L.; Petrosino, J. F.; Bode, L.; et al. Human milk oligosaccharide DSLNT and gut microbiome in preterm infants predicts necrotising enterocolitis. Gut 2021, 70 (12), 2273.
(7) Rogers, G. N.; Pritchett, T. J.; Lane, J. L.; Paulson, J. C. Differential sensitivity of human, avian, and equine influenza-A viruses to a glycoprotein inhibitor of infection-selection of receptor specific variants. Virology 1983, 131 (2), 394.
(8) Chutipongtanate, S.; Morrow, A. L.; Newburg, D. S. Human milk oligosaccharides: Potential applications in COVID-19. Biomedicines 2022, 10 (2).
(9) Smith, H.; Grant, S.; Parker, J.; Murphy, R. Yeast cell wall mannan rich fraction modulates bacterial cellular respiration potentiating antibiotic efficacy. Sci. Rep. 2020, 10 (1). Oba, S.; Sunagawa, T.; Tanihiro, R.; Awashima, K.; Sugiyama, H.; Odani, T.; Nakamura, Y.; Kondo, A.; Sasaki, D.; Sasaki, K. Prebiotic effects of yeast mannan, which selectively promotes Bacteroides thetaiotaomicron and Bacteroides ovatus in a human colonic microbiota model. Sci. Rep. 2020, 10 (1).
(10) Moya-Gonzálvez, E. M.; Rubio-del-Campo, A.; Rodríguez-Díaz, J.; Yebra, M. J. Infant-gut associated Bifidobacterium dentium strains utilize the galactose moiety and release lacto-N-triose from the human milk oligosaccharides lacto-N-tetraose and lacto-N-neotetraose. Sci. Rep. 2021, 11 (1).
(11) Chen, X. D.; Xu, L.; Jin, L.; Sun, B.; Gu, G. F.; Lu, L. L.; Xiao, M. Efficient and regioselective synthesis of β-GalNAc/GlcNAc-lactose by a bifunctional transglycosylating β-N-acetylhexosaminidase from Bifidobacterium bifidum. Appl. Environ. Microbiol. 2016, 82 (18), 5642.
(12) Schmolzer, K.; Weingarten, M.; Baldenius, K.; Nidetzky, B. Glycosynthase Principle Transformed into Biocatalytic Process Technology: Lacto-N-triose II Production with Engineered exo-Hexosaminidase. ACS Catal. 2019, 9 (6), 5503.
(13) Noguchi, M.; Tanaka, T.; Gyakushi, H.; Kobayashi, A.; Shoda, S. Efficient synthesis of sugar oxazolines from unprotected N-acetyl-2-amino sugars by using chloroformamidinium reagent in water. J. Org. Chem. 2009, 74 (5), 2210.
(14) McArthur, J. B.; Yu, H.; Chen, X. A bacterial β-1-3-galactosyltransferase enables multigram-scale synthesis of human milk lacto-N-tetraose (LNT) and its fucosides. ACS Catal. 2019, 9 (12), 10721.
(15) Tsai, T. W.; Fang, J. L.; Liang, C. Y.; Wang, C. J.; Huang, Y. T.; Wang, Y. J.; Li, J. Y.; Yu, C. C. Exploring the synthetic application of Helicobacter pylori α-1,3/4-fucosyltransferase FucTIII toward the syntheses of fucosylated human milk glycans and Lewis antigens. ACS Catal. 2019, 9 (12), 10712.
(16) Zivkovic, A. M.; German, J. B.; Lebrilla, C. B.; Mills, D. A. Human Milk Glycobiome and its Impact on the Infant Gastrointestinal Microbiota. Proc. Natl. Acad. Sci. U.S.A. 2011, 108, 4653.
(17) Westwood, S.; Yamazaki, T.; Huang, T.; Garrido, B.; Ün, I.; Zhang, W.; Martos, G.; Stoppacher, N.; Saito, T.; Wielgosz, R. Development and Validation of a Suite of Standards for the Purity Assignment of Organic Compounds by Quantitative NMR Spectroscopy. Metrologia 2019, 56 (6).

### CONCLUDING REMARKS

Although the present subject disclosure has been described in connection with preferred embodiments thereof, it will be appreciated by those skilled in the art that additions, deletions, modifications, and substitutions not specifically described may be made without departing from the spirit and scope of the subject disclosure. Terminology used herein should not be construed as being “means-plus-function” language unless the term “means” is expressly used in association therewith.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A human milk oligosaccharide (HMO) analog, comprising:
N-acetylmannosamine (ManNAc) and lactose (Lac).

2. The human milk oligosaccharide (HMO) analog of claim 1, comprising the structure as follows:

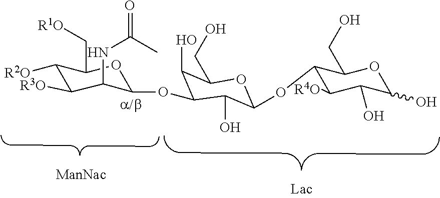

3. The human milk oligosaccharide (HMO) analog of claim 2,
wherein:
R¹ is H or α2,6-Neu5Ac;
R² is H, α1,4-Fuc, β1,4-Gal or β1,4-Gal-α2,3-Neu5Ac;
R³ is H, α1,3-Fuc, β1,3-Gal or β1,3-Gal-α2,3-Neu5Ac; and
R⁴ is H or α1,3-Fuc.

4. The human milk oligosaccharide (HMO) analog of claim 3,
wherein the ManNAc constituent cannot be directly and
simultaneously fucosylated and sialylated or multifucosylated.

5. The human milk oligosaccharide (HMO) analog of claim 3,
wherein analogs comprise one of the analogs depicted in FIG. 7A.

6. A method comprising formulating the human milk
oligosaccharide (HMO) analog of claim 2 using the scheme depicted in FIG. 2.
